(19) United States
(12) Patent Application Publication
(10) Pub. No.: US 2026/0097145 A1
(43) Pub. Date: Apr. 9, 2026

(54) MULTIFUNCTIONAL AROMA DIFFUSER

(71) Applicant: LONGWEI ZHANG, Dongguan (CN)

(72) Inventor: LONGWEI ZHANG, Dongguan (CN)

(21) Appl. No.: 18/915,601

(22) Filed: Oct. 15, 2024

(30) Foreign Application Priority Data
Oct. 8, 2024 (CN) 202422420028.2

(51) Int. Cl. A61L 9/14

(52) U.S. Cl. CPC A61L 9/14; A61L 2209/133; A61L 2209/135

(57) ABSTRACT

Disclosed is a multifunctional aroma diffuser. A housing is provided with an atomizing mounting part, and the atomizing mounting part includes at least two bottle body fixing sites, at least two air nozzles, and at least two atomizing fixing sites. At least two of the atomizing devices are arranged and correspond to the two atomizing fixing sites respectively. Either of the atomizing devices includes an atomizing shell, an atomizing sheet and an atomizing elastic member, and at least one atomizing hole is formed in the atomizing sheet. The bottle body fixing sites can be configured for fixing aroma bottles. By increasing the bottle body fixing sites in the atomizing mounting part, a plurality of the aroma bottles can be fixed, and only one aroma diffuser is needed to release various scents of aroma from a plurality of the aroma bottles simultaneously.

### CROSS-REFERENCE TO RELATED APPLICATIONS

This application claims priority to Chinese Patent Application No. 202422420028.2, filed on Oct. 8, 2024, which is hereby incorporated by reference in its entirety.

### TECHNICAL FIELD

The present disclosure belongs to the field of aroma diffusers, and particularly relates to a multifunctional aroma diffuser.

### BACKGROUND

An aroma diffuser emits fragrance through evaporation of essential oil, which can optimize scent of air, improve ambient air and keep fresh the air in an entire environment. The aroma diffuser includes a housing, an atomizing sheet, and a vibration device. The housing is provided with a mounting site for placing the atomizing sheet and the vibration device. The atomizing sheet and the vibration device are mounted in the housing. Through high-frequency oscillation of the atomizing sheet, a mixture of essential oil and water is atomized and diffused into air, such that an entire space is filled with fragrance.

However, an aroma diffuser in the prior art cannot release aroma of various scents simultaneously, and a plurality of aroma diffusers are needed to release aroma of various scents, such that a demand for such aroma diffusers increases. Further, the aroma diffusers in the prior art are relatively single in functions, and are not suitable for some indoor scenarios of use.

Moreover, an aroma diffuser in the prior art has defects. The vibration device and the atomizing sheet need to be positioned in the housing, such that when the housing structure is small, assembly time and costs are increased due to difficult and low-efficiency assembly and positioning of the vibration device and the atomizing sheet in the housing.

### SUMMARY

### (1) Technical Problems to be Solved

In view of the defects in the prior art, an objective of the present disclosure is to provide a multifunctional aroma diffuser so as to solve the technical problems of the prior art, including failure to release aroma of various scents simultaneously through an aroma diffuser and low-efficiency assembly thereof.

### (2) Technical Solutions

A multifunctional aroma diffuser includes a housing and atomizing devices. The housing is provided with an atomizing mounting part, and the atomizing mounting part includes at least two bottle body fixing sites, at least two air nozzles, and at least two atomizing fixing sites. At least two of the atomizing devices are arranged and correspond to the two atomizing fixing sites respectively. Either of the atomizing devices includes an atomizing shell, an atomizing sheet and an atomizing elastic member, at least one atomizing hole is formed in the atomizing sheet, and the atomizing shell is provided with an atomizing snap-fit site corresponding to the corresponding atomizing fixing site.

Further, the atomizing shell is provided with an atomizing chamber, one end of the atomizing chamber is provided with a mist discharge hole, and the other end thereof is provided with a stop block.

Further, the housing includes a first housing body and a second housing body. Either of the atomizing fixing sites includes a vertical fixing plate, a horizontal fixing plate and a bottleneck limiting block, where the bottleneck limiting block is provided with an outflow hole.

Further, a recess is formed in each of the horizontal fixing plates, a mounting cavity is formed between the vertical fixing plates and the horizontal fixing plates, and the atomizing snap-fit site includes snap latches.

Further, the first housing body is provided with a control circuit device, the atomizing sheet is electrically connected to the control circuit device, the atomizing sheet is provided with an atomizing site, the atomizing hole is formed in the atomizing site, and the atomizing site comprises a concave surface and a convex surface, wherein the concave surface is arranged on a side of the outflow hole, and the convex surface is arranged on a side of the mist discharge hole.

Further, the bottle body fixing site includes an fixing bracket, the fixing bracket is recessed downward along its upper end to form an avoidance hole that matches an aroma bottle in a shape, the fixing bracket is provided with a fixing latch at an upper end of the avoidance hole, and the aroma bottle is provided with a positioning groove that fits the fixing latch.

Further, the aroma bottle includes a bottle stopper and a cotton core, an upper end of the cotton core corresponds to a position of the outflow hole, and a lower end of the cotton core passes through the bottle stopper and enters the aroma bottle, and makes contact with a bottom of the aroma bottle.

Further, a detachable outer cover is arranged on the first housing body.

Further, a side of the control circuit device is electrically connected to a plug, a rotating member is provided on a side of the plug, the first housing body is provided with an upper rotating limiting groove, and the second housing body is provided with a lower rotating limiting groove.

Further, the lower rotating limiting groove and/or the upper rotating limiting groove is provided with a rotating position groove, the rotating member is provided with at least one rotating stop block and one snapping block, and the rotating position groove includes a horizontal position groove, an inclined position groove, and a vertical position groove.

### (3) Beneficial Effects

Compared with the prior art, the present disclosure has the beneficial effects as follows:

The atomizing mounting part of the present disclosure includes at least two bottle body fixing sites, at least two air nozzles, and at least two atomizing fixing sites. At least two of the atomizing devices are arranged and correspond to the two atomizing fixing sites respectively. Either of the atomizing devices includes an atomizing shell, an atomizing sheet and an atomizing elastic member, at least one atomizing hole is formed in the atomizing sheet, and the atomizing elastic member and the atomizing sheet are mounted in the atomizing shell. The bottle body fixing sites can be configured for fixing aroma bottles, and either of the atomizing devices is arranged between the bottle body fixing site and the air nozzle. The atomizing shell is provided with an atomizing snap-fit site corresponding to the atomizing fixing site. By increasing the bottle body fixing sites in the atomizing mounting part, a plurality of the aroma bottles can be fixed, without need to match a plurality of aroma diffusers with a plurality of aroma bottle sites in one-to-one correspondence, that is, only one aroma diffuser is needed to release various scents of aroma from a plurality of the aroma bottles simultaneously, thereby reducing the number of aroma diffusers needed and reducing use costs.

### BRIEF DESCRIPTION OF DRAWINGS

FIG. 1 is an exploded view of an aroma diffuser of the present disclosure.

FIG. 2 is an exploded view of an atomizing mounting part and an atomizing device of the present disclosure.

FIG. 3 is a sectional view of an aroma bottle and an aroma diffuser fixed to each other according to the present disclosure.

FIG. 4 is an exploded view I of an aroma bottle and an aroma diffuser fixed to each other according to the present disclosure.

FIG. 5 is an exploded view II of an aroma bottle and an aroma diffuser fixed to each other according to the present disclosure.

FIG. 6 is a sectional view of the present disclosure when a plug corresponds to a parallel socket.

FIG. 7 is a sectional view of the present disclosure when a plug corresponds to a vertical socket.

Reference numerals in the figures: 1. first housing body; 11. bottle body fixing site; 111. fixing bracket; 112. avoidance hole; 113. fixing latch; 12. atomizing fixing site; 121. vertical fixing plate; 122. horizontal fixing plate; 123. bottleneck limiting block; 124. outflow hole; 125. recess; 13. lower rotating limiting groove; 2. second housing body; 21. air nozzle; 22. upper rotating limiting groove; 3. atomizing device; 31. atomizing shell; 311. atomizing chamber; 312. mist discharge hole; 313. stop block; 32. atomizing sheet; 321. atomizing site; 322. concave surface; 323. convex surface; 33. atomizing elastic member; 34. atomizing hole; 35. snap latch; 4. control circuit device; 41. control switch; 5. aroma bottle; 51. positioning groove; 52. bottle stopper; 53. cotton core; 7. plug; 8. rotating member; 81. rotating stop block; 82. snapping block; 9. rotating position groove; 91. horizontal position groove; 92. inclined position groove; and 93. vertical position groove.

### DESCRIPTION OF EMBODIMENTS

The technical solutions in the examples of the present disclosure will be clearly and completely described below with reference to the accompanying drawings in the examples of the present disclosure.

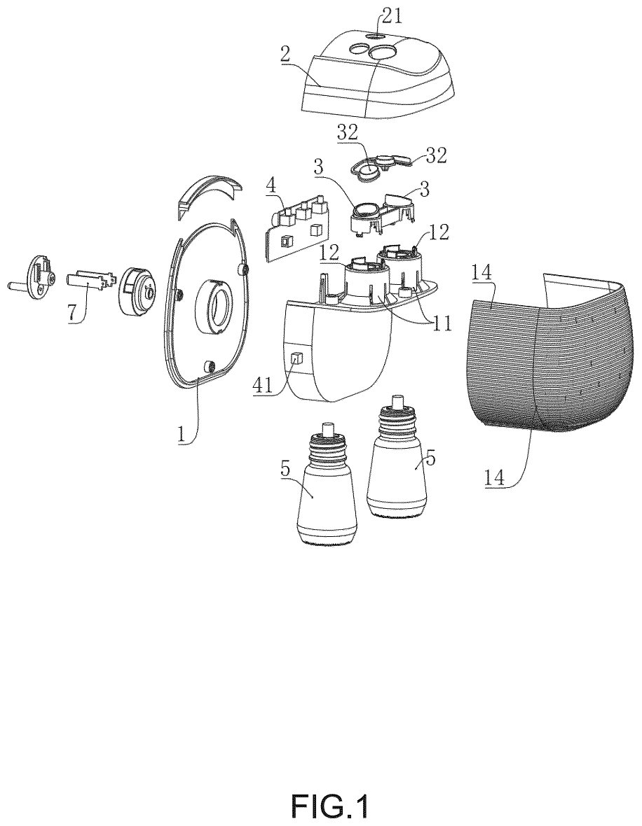

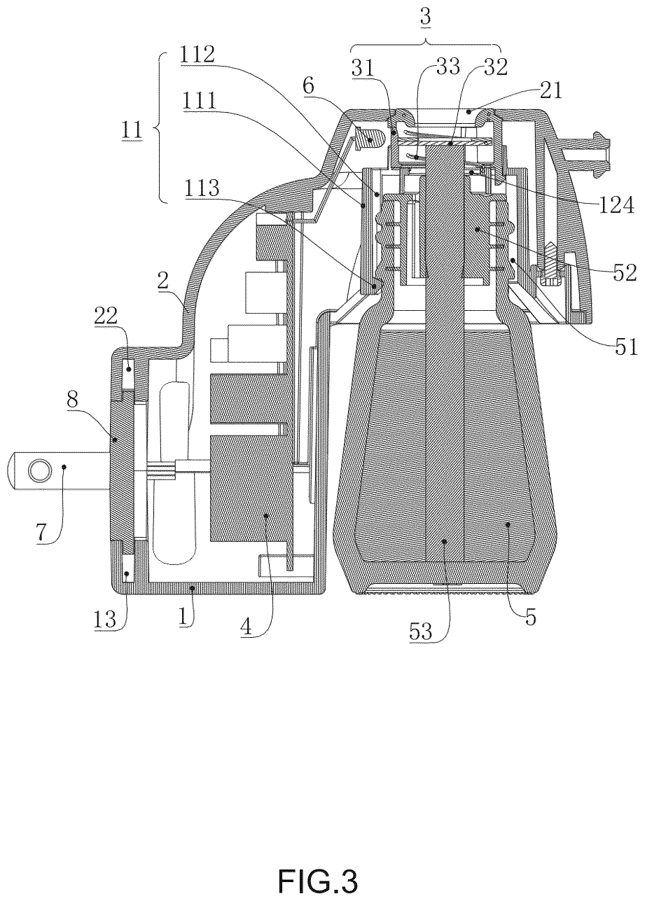

Refer to FIGS. 1-7.

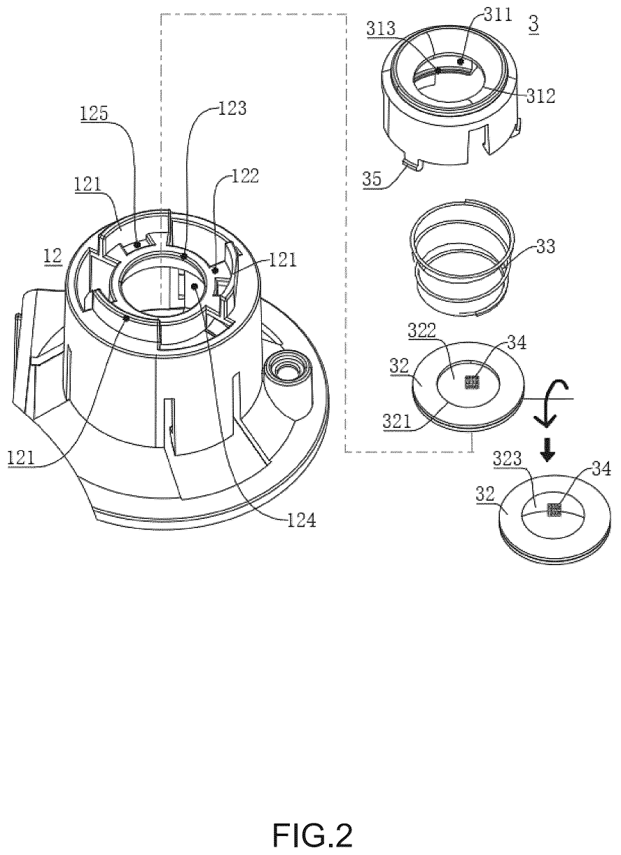

As illustrated in FIGS. 1 and 2, a multifunctional aroma diffuser of the present disclosure includes a housing and atomizing devices 3. The housing is provided with an atomizing mounting part, and the atomizing mounting part includes at least two bottle body fixing sites 11, at least two air nozzles 21, and at least two atomizing fixing sites 12. At least two of the atomizing devices 3 are arranged and correspond to the two atomizing fixing sites 12 respectively. Either of the atomizing devices 3 includes an atomizing shell 31, an atomizing sheet 32 and an atomizing elastic member 33, at least one atomizing hole 34 is formed in the atomizing sheet 32, and the atomizing elastic member 33 and the atomizing sheet 32 are mounted in the atomizing shell 31. The bottle body fixing sites 11 can be configured for fixing aroma bottles 5, and either of the atomizing devices 3 is arranged between the bottle body fixing site 11 and the air nozzle 21. The atomizing shell 31 is provided with an atomizing snap-fit site corresponding to the atomizing fixing site 12. By increasing the bottle body fixing sites 11 in the atomizing mounting part, a plurality of the aroma bottles 5 can be fixed, without need to match a plurality of aroma diffusers with a plurality of aroma bottle sites in one-to-one correspondence, that is, only one aroma diffuser is needed to release various scents of aroma from a plurality of the aroma bottles simultaneously, thereby reducing the number of aroma diffusers needed and reducing use costs.

FIGS. 2-7 and a following description of the aroma diffuser all illustrate assembly steps of only one of the atomizing devices 3. Because assembly steps of the other atomizing devices 3 are the same, the assembly steps of the other atomizing devices will not be described or illustrated separately.

During assembly, the atomizing sheet 32, the atomizing elastic member 33, and the atomizing shell 31 are assembled outside the housing to form the atomizing device 3. Then the entire atomizing device 3 is inserted into the housing, and the atomizing snap-fit site is snap-fitted onto the atomizing fixing site 12 to achieve mounting and fixation of the atomizing device 3 to the housing. A simple structure and the assembly outside the housing avoid positioning of the atomizing sheet 32 and the atomizing elastic member 33 with the housing, thereby effectively improving assembly efficiency and reducing assembly time and costs. When in use, the aroma bottle 5 is placed at the bottle body fixing site 11, and the atomizing sheet 32 corresponds to an opening of the aroma bottle 5. When the atomizing elastic member 33 and the atomizing sheet 32 vibrate at a high frequency at the opening of the aroma bottle 5, liquid in the aroma bottle 5, after passing through the atomizing hole 34, is mixed with a water mist formed by air and then discharged from the air nozzle 21, to achieve uniform atomization and diffusion of the diffuser liquid.

As illustrated in FIG. 1, the housing includes a first housing body 1 and a second housing body 2, where a detachable outer cover 14 is located outside the second housing body 2, and the outer cover 14 can be provided with various patterns. According to actual needs of users, the outer cover 14 can be assembled together with the second housing body 2 to make the aroma diffuser look more beautiful, and can also be removed from the second housing body 2, thereby facilitating flexible use.

As illustrated in FIG. 1, the first housing body 1 is provided with a control switch 41, and the control switch 41 is electrically connected to a control circuit device 4. In this example, the control switch 41 has three gear positions: fast, medium, and slow. Through adjustment of the control switch 41, the atomizing sheet 32 can vibrate at a fast, medium or slow speed, to achieve a desired atomizing effect.

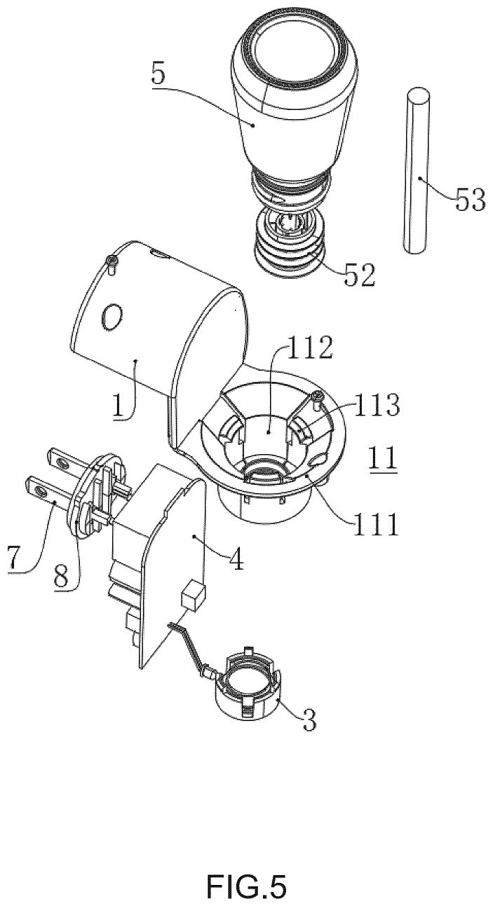

As illustrated in FIGS. 5-7, the bottle body fixing site 11 and the atomizing fixing site 12 are arranged on the first housing body 1, and the air nozzle 21 is arranged on the second housing body 2. The atomizing fixing site 12 is located at an end of the bottle body fixing site 11, and the atomizing fixing site 12 includes vertical fixing plates 121, horizontal fixing plates 122, and a bottleneck limiting block 123. An outflow hole 124 is formed in the bottleneck limiting block 123, and a recess 125 is formed in each of the horizontal fixing plates 122. In this example, a mounting cavity that is structurally compatible with the atomizing shell 31 is formed between the vertical fixing plates 121 (three ones) and the horizontal fixing plates 122 (three ones). The atomizing snap-fit site includes snap latches 35. The atomizing shell 31 of the atomizing device 3 is aligned with the mounting cavity, and the snap latches 35 are aligned with and pressed into the recesses 125. In this case, the snap latches 35 are snap-fitted into the recesses 125, such that the atomizing device 3 is fixed at a position of the mounting cavity in a snap-fitting manner. A position of the atomizing sheet 32 corresponds to the outflow hole 124 of the bottleneck limiting block 123. When disassembly, replacement or maintenance is needed, a user only needs to pull out the atomizing device 3 in a direction away from the atomizing fixing site 12 to disengage the snap latches 35 from the recesses 125. Due to simple disassembly and assembly steps, each user can easily complete the assembly and replacement.

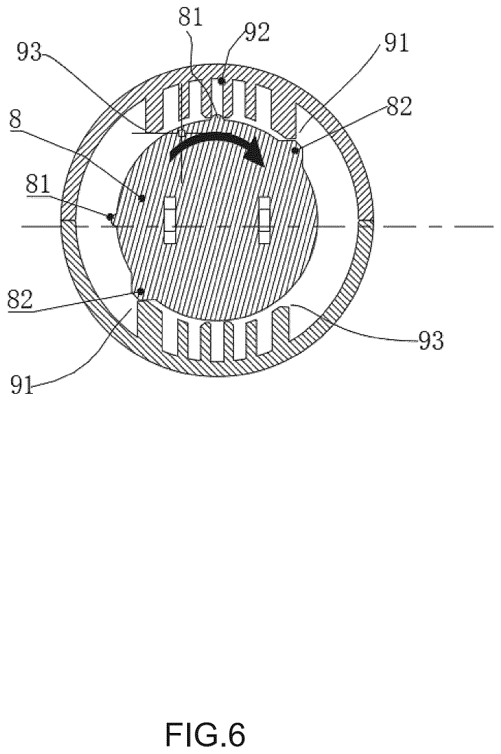

As illustrated in FIGS. 5-6, the atomizing shell 31 is provided with an atomizing chamber 311, and the atomizing sheet 32 and the atomizing elastic member 33 are placed inside the atomizing chamber 311. One end of the atomizing chamber 311 is provided with a mist discharge hole 312, and the other end thereof is provided with a stop block 313, where the stop block 313 abuts against and limits the atomizing sheet 32 in the atomizing chamber 311, to prevent the atomizing sheet 32 and the atomizing elastic member 33 from detaching from the atomizing chamber 311, thereby avoiding failures of atomizing.

Specifically, the position of the atomizing sheet 32 corresponds to the outflow hole 124 of the bottleneck limiting block 123. The first housing body 1 is provided with the control circuit device 4, and the atomizing sheet 32 is electrically connected to the control circuit device 4. The atomizing sheet 32 is provided with an atomizing site 321, and the atomizing hole 34 is formed in the atomizing site 321. The atomizing site 321 includes a concave surface 322 and a convex surface 323, where the concave surface 322 is arranged on a side of the outflow hole 124, and the convex surface 323 is arranged on a side of the mist discharge hole 312. Under control of the control circuit device 4, high-frequency oscillation caused by the atomizing sheet 32 results in that essential oil in the aroma bottle 5 is decomposed into nano-scale cold mist with a diameter of 0.1-5 microns at a position of the concave surface 322, and then the cold mist is diffused in surrounding air gradually through the convex surface 323, such that the air is filled with fragrance. Arrangement of the concave surface 322 and the convex surface 323 enhances the atomizing effect, broadens a dispersion range, and improves atomizing efficiency.

Specifically, the aroma bottle 5 includes a bottle stopper 52 and a cotton core 53. An upper end of the cotton core 53 corresponds to a position of the outflow hole 124 and is in contact with the concave surface 322 of the atomizing sheet 32, and a lower end of the cotton core 53 passes through the bottle stopper 52 and enters the aroma bottle 5, and makes contact with a bottom of the aroma bottle 5, such that the essential oil inside the aroma bottle 5 can be continuously supplied upward through an adsorption action of the cotton core 53. The atomizing sheet 32 and the concave surface 322, in the high-frequency oscillation state, press the cotton core 53, and water molecules and dissolved essential oil inside the cotton core 53 are decomposed into the nano-scale cold mist with a diameter of 0.1-5 microns at the position of the concave surface 322, and the cold mist is then diffused into the surrounding air through the convex surface 323.

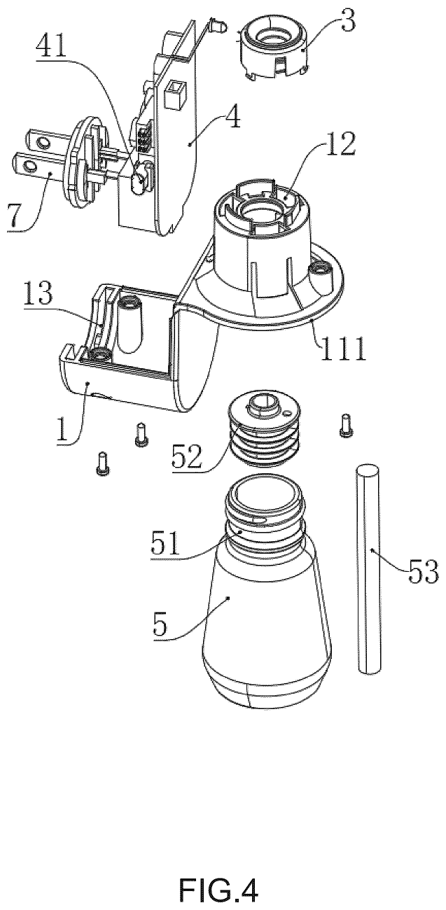

As illustrated in FIGS. 4-5, the bottle body fixing site 11 includes a fixing bracket 111, the fixing bracket 111 is recessed downward along its upper end to form an avoidance hole 112 that matches the aroma bottle 5 in a shape. The aroma bottle 5 is detachably mounted in the avoidance hole 112, the fixing bracket 111 is provided with a fixing latch 113 at an upper end of the avoidance hole 112, and the aroma bottle 5 is provided with a positioning groove 51 that fits the fixing latch 113. When necessary to add the essential oil, the user can directly remove the aroma bottle 5 from the avoidance hole 112 and reinstall the aroma bottle 5 in the avoidance hole 112 after adding the essential oil. Cooperation between the fixing latch 113 and the positioning groove 51 facilitates positioning and fixing of the aroma bottle 5, and prevents the aroma bottle 5 from being loosened.

As illustrated in FIG. 1, a side of the control circuit device 4 is electrically connected to a plug 7, a rotating member 8 is provided on a side of the plug 7, and the plug 7 is mounted on the rotating member 8. The first housing body 1 is provided with an upper rotating limiting groove 22 that corresponds to the rotating member 8, and the second housing body 2 is provided with a lower rotating limiting groove 13 that corresponds to the rotating member 8. The rotating member 8 is simultaneously snap-fitted into both the lower rotating limiting groove 13 and the upper rotating limiting groove 22 to achieve fixation between the second housing body 2 and the first housing body 1, so as to fix the plug 7 on both the second housing body 2 and the first housing body 1.

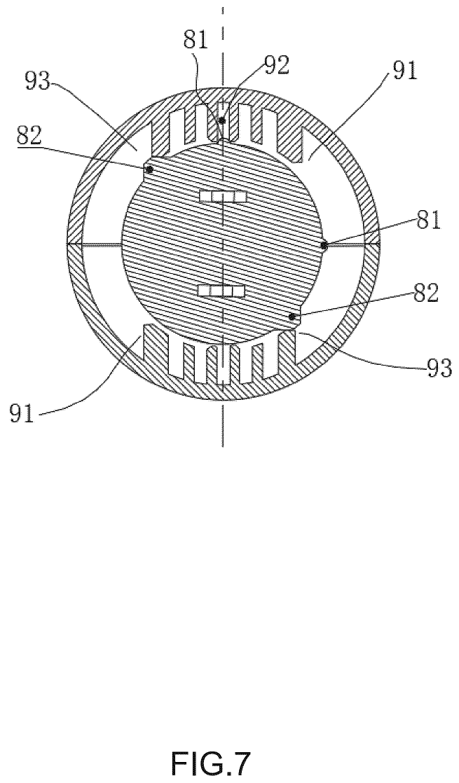

As illustrated in FIGS. 6-7, the lower rotating limiting groove 13 and/or the upper rotating limiting groove 22 is provided with a rotating position groove 9. The rotating member 8 is provided with at least one rotating stop block 81 and one snapping block 82. The rotating position groove 9 includes a horizontal position groove 91, an inclined position groove 92, and a vertical position groove 93. The rotating stop block 81 is snap-fitted into the inclined position groove 92. When two jacks of a socket are horizontally arranged, the rotating member 8 can be rotated between the first housing body 1 and the second housing body 2 such that the snapping block 82 abuts against the horizontal position groove 91, thereby ensuring that the plug 7 and the two jacks are simultaneously aligned on a same horizontal line for easy insertion. When the two jacks of the socket are vertically arranged, the rotating member 8 can be rotated between the first housing body 1 and the second housing body 2 such that the snapping block 82 abuts against the vertical position groove 93, thereby ensuring that the plug 7 and the two jacks are simultaneously aligned on a same vertical line for easy insertion. The arrangement of the rotating position groove 9 ensures that the plug 7 adapts to different socket arrangement modes, thereby enhancing flexibility of use and effectively optimizing user experience.

For those skilled in the art, it is apparent that the present disclosure is not limited to the details of the above-mentioned exemplary examples, and the present disclosure can be implemented in other specific forms without departing from the spirit or basic features of the present disclosure. Therefore, no matter from which point of view, the examples should all be regarded as exemplary and non-limiting. The scope of the present disclosure is defined by the appended claims rather than the above-mentioned description, and therefore it is intended that all changes which fall within the meaning and scope of equivalency of the claims are embraced in the present disclosure. Any reference numeral in the claims should not be construed as limiting the related claims.

In addition, it should be understood that although the present description is described according to the embodiments, not each embodiment only contains an independent technical solution, and the specification manner of the description is only for clarity. Those skilled in the art should take the description as a whole, and the technical solutions in the examples can also be appropriately combined to form other embodiments that can be understood by those skilled in the art.

What is claimed is:

1. A multifunctional aroma diffuser, comprising a housing and atomizing devices, wherein the housing is provided with an atomizing mounting part, and the atomizing mounting part comprises at least two bottle body fixing sites, at least two air nozzles, and at least two atomizing fixing sites; at least two of the atomizing devices are arranged and correspond to the two atomizing fixing sites respectively; and either of the atomizing devices comprises an atomizing shell, an atomizing sheet and an atomizing elastic member, at least one atomizing hole is formed in the atomizing sheet, and the atomizing shell is provided with an atomizing snap-fit site corresponding to the corresponding atomizing fixing site.

2. The multifunctional aroma diffuser according to claim 1, wherein the atomizing shell is provided with an atomizing chamber, one end of the atomizing chamber is provided with an mist discharge hole, and the other end thereof is provided with a stop block.

3. The multifunctional aroma diffuser according to claim 2, wherein the housing comprises a first housing body and a second housing body, either of the atomizing fixing sites comprises a vertical fixing plate, a horizontal fixing plate and a bottleneck limiting block, and the bottleneck limiting block is provided with an outflow hole.

4. The multifunctional aroma diffuser according to claim 3, wherein a recess is formed in each of the horizontal fixing plates, a mounting cavity is formed between the vertical fixing plates and the horizontal fixing plates, and the atomizing snap-fit site comprises snap latches.

5. The multifunctional aroma diffuser according to claim 4, wherein the first housing body is provided with a control circuit device, the atomizing sheet is electrically connected to the control circuit device, the atomizing sheet is provided with an atomizing site, the atomizing hole is formed in the atomizing site, and the atomizing site comprises a concave surface and a convex surface, wherein the concave surface is arranged on a side of the outflow hole, and the convex surface is arranged on a side of the mist discharge hole.

6. The multifunctional aroma diffuser according to claim 5, wherein the bottle body fixing site comprises an fixing bracket, the fixing bracket is recessed downward along its upper end to form an avoidance hole that matches an aroma bottle in a shape, the fixing bracket is provided with a fixing latch at an upper end of the avoidance hole, and the aroma bottle is provided with a positioning groove that fits the fixing latch.

7. The multifunctional aroma diffuser according to claim 6, wherein the aroma bottle comprises a bottle stopper and a cotton core, an upper end of the cotton core corresponds to a position of the outflow hole, and a lower end of the cotton core passes through the bottle stopper and enters the aroma bottle, and makes contact with a bottom of the aroma bottle.

8. The multifunctional aroma diffuser according to claim 7, wherein a detachable outer cover is arranged on the first housing body.

9. The multifunctional aroma diffuser according to claim 8, wherein a side of the control circuit device is electrically connected to a plug, a rotating member is provided on a side of the plug, the first housing body is provided with an upper rotating limiting groove, and the second housing body is provided with a lower rotating limiting groove.

10. The multifunctional aroma diffuser according to claim 9, wherein the lower rotating limiting groove and/or the upper rotating limiting groove is provided with a rotating position groove, the rotating member is provided with at least one rotating stop block and one snapping block, and the rotating position groove comprises a horizontal position groove, an inclined position groove, and a vertical position groove.
